(19) United States
(12) Patent Application Publication
(10) Pub. No.: US 2016/0256147 A1
(43) Pub. Date: Sep. 8, 2016

(54) RETRACTOR FOR VIDEO-ASSISTED THORACOSCOPIC SURGERY

(71) Applicants: Seung Heum Jeong, Seoul (KR); Hui Cheol Yang, Gyeonggi-do (KR)

(72) Inventors: Seung Heum Jeong, Seoul (KR); Hui Cheol Yang, Gyeonggi-do (KR)

(21) Appl. No.: 15/061,197

(22) Filed: Mar. 4, 2016

(30) Foreign Application Priority Data
Mar. 5, 2015 (KR) 10-2015-0031026

(51) Int. Cl. A61B 17/02

(52) U.S. Cl. CPC A61B 17/02

(57) ABSTRACT

A retractor for video-assisted thoracoscopic surgery. The retractor includes a core provided with a first bent portion, a second bent portion vertically extending from the first bent portion, a third bent portion horizontally extending from the second bent portion to correspond to the first bent portion, the core having an insert core part on one side thereof and a hook core part on the other side, a silicon body configured to encapsulate a circumference of the core, a first hook part extending from one side of the silicon body to encapsulate the insert core part of the core, and a second hook part extending from the other side of the silicon body to encapsulate the hook core part of the core.

### CROSS-REFERENCE TO RELATED APPLICATION

This application claims priority to and the benefit of Korean Patent Application No. 10-2015-0031026, filed on Mar. 5, 2015, the disclosure of which is incorporated herein by reference in its entirety.

### FIELD OF THE INVENTION

The present invention relates to a retractor for video-assisted thoracoscopic surgery, and more particularly, to a retractor for video-assisted thoracoscopic surgery capable of being bent so that an area for surgery of a patient is easily hooked and pulled by the retractor to secure a view for the video-assisted thoracoscopic surgery and the retractor gently contacts the area for surgery when the area for surgery is pulled by a silicon body thereof.

### BACKGROUND OF THE INVENTION

In general, lung surgery is largely classified into thoracotomies in which a side of the chest is opened and spread between the ribs, and thoracoscopic surgeries in which three to four small holes are pierced between the ribs, and a thoracoscope and surgical instruments are inserted into the holes to perform lung surgery by using a view through a camera.

Recently, the percentage of thoracoscopic surgeries has been gradually increasing due to demand for minimally invasive surgeries and advances in technology.

Surgeries performed using thoracoscopes, for example, include pneumonectomies, esophagectomies, tracheobronchoplasties, mediastinal lymph node dissections, and mediastinal neoplasm resections.

When a surgery is performed on a patient with problems in the thorax, a retractor is commonly used as a tool for securing a view of the area for surgery.

A retractor refers to one type of surgical instrument which hooks and pulls a patient's area for surgery to secure a view for surgery with respect to the surgical part. A retractor for video-assisted thoracoscopic surgery is characterized in that an instrument is inserted through a hole (usually, the hole has a diameter of about 1 cm to about 3 cm) unlike a retractor for thoracotomy, and the retractor has a thin and long body because the retractor has to be manipulated from outside the patient's body.

In a related art, since a laparoscopic retractor is used for thoracoscopic surgery, the shape of a retracting part is not suitable for thoracic surgery. Also, since the laparoscopic retractor is not flexible, it is difficult to secure a view of everywhere in the thorax through narrow spaces between the ribs.

As illustrated in FIG. 1, a retractor according to the related art includes a long stick-shaped body 2 and a circular hook part 1 disposed on one end of the long stick-shaped body 2. In a state where the long stick-shaped body 2 is grasped, the circular hook part 1 is hooked on an area for surgery to pull or push the area for surgery so that a view for surgery is secured.

However, as illustrated in FIG. 1, since the retractor according to the related art has the hook part 1 only on the one end of the long stick-shaped body 2 and may only be used in the straight shape thereof, there are limits to an operation in which an area for surgery is hooked and pulled by using the retractor. Also, when the angle between an instrument entering part and a target point is not correct, the usefulness of an instrument is compromised and as a result, a view for surgery may not be properly secured. Difficulties in securing a view for surgery mean that surgeries related the most important organs (the lungs, the pulmonary artery, the pulmonary vein, the esophagus, the trachea/bronchus, and the heart) in the thorax of the human body may result in accidents such as hemorrhaging.

For example, in order to more safely and accurately secure a view for thoracoscopic surgery, retractor hook parts having respectively different sizes depending on target areas are imperative. However, when various kinds of retractors are used, it is inconvenient to change retractors during surgery, and since various kinds of retractors have to be prepared, there are problems in the purchasing costs of the retractors and storing and sterilizing the retractors.

Also, since the retractor according to the related art has the hook part 1 having a circular shape, a phenomenon in which the hook part 1 slides off the area for surgery while attempting to hook and pull the area for surgery may frequently occur to hinder the delicate surgery.

For example, the retractor is ineffective and hazardous when retracting a bulky part like lung tissue, a cylindrical organ like the esophagus, or a blood vessel that has the potential to rupture when strongly pushed.

Moreover, since the long stick-shaped body 2 of the retractor according to the related art is not bent, views for surgery from various angles may not be secured by using the retractor.

Also, since the long stick-shaped body 2 has a straight shape, the retractor inevitably comes into the view of the camera when compared to a bent-type or curved-type body and collides with other instruments.

Referring to the related art, a retractor for lumbar surgery is disclosed in Korean Utility Registration No. 20-0248090. The retractor for lumbar surgery includes: a rack having a predetermined length; a fixed lever of which one side is fixed to the rack, the fixed lever having a mounting groove defined in the other side thereof to allow a fixing-side blade to be mounted; a moving lever of which one side is movably disposed on a rack opposite to the fixed lever by interposing a pinion between the rack and the moving lever, the moving lever having a mounting groove defined in the other side thereof to allow a moving-side blade to be mounted; a handle connected to a rotation shaft integrated with the pinion of the moving lever through a yoke on one side thereof; and a latch of which the front end is coupled to the rack to prevent the moving lever from moving toward the fixed lever on the rack when the handle is not manipulated.

According to the related art, the retractor may maintain a distance between the fixed-side blade and the moving-side blade which hold muscles in a predetermined state. Also, since the retractor is applied to pelvises of various sizes, a surgery assistant may spread the muscles around the area for surgery by using other instruments, or the retractor may be appropriately used to correspond to the size of the pelvis, and thus a stable and broad view of the surgery may be secured on the lumbar area for surgery.

However, since the related art discloses a technology for a retractor for lumbar surgery, the retractor, due to the structure thereof, may not be used in thoracoscopic surgery as a unit for pulling or spreading an area for surgery to secure a view for surgery.

Also, in the retractor according to the related art, since the hook part that pulls the area for surgery is formed of a hard material, there is the frequently occurring issue that when the area for surgery is hooked and pulled, the hooked area for surgery may tear or be otherwise damaged.

### RELATED DOCUMENTS

(Patent document 1) Korean Utility Registration No. 20-0248090 (Sep. 13, 2001)
(Patent document 2) Korean Patent Registration No. 10-0879798 (Jan. 14, 2009)

### SUMMARY OF THE INVENTION

The present invention provides a retractor for video-assisted thoracoscopic surgery, which is capable of safely firmly hooking and pulling organs in thorax when video-assisted thoracoscopic surgery is performed, and which has hook parts respectively having sizes different from each other on both sides on a bendable body thereof to improve usability and convenience thereof.

In an embodiment of the present invention, a retractor for video-assisted thoracoscopic surgery includes: a core provided with a first bent portion, a second bent portion vertically extending from the first bent portion, a third bent portion horizontally extending from the second bent portion to correspond to the first bent portion, the core having an insert core part on one side thereof and a hook core part on the other side; a silicon body configured to encapsulate a circumference of the core; a first hook part extending from one side of the silicon body to encapsulate the insert core part of the core; and a second hook part extending from the other side of the silicon body to encapsulate the hook core part of the core, wherein the first hook part includes: first and second arc-shaped portions respectively extending from the one side of the silicon body to symmetrical to each other, wherein each of the first and second arc-shaped portions extends in a tapered shape to allow the first hook part to be easily hooked on an area for surgery; and a third arc-shaped portion that connects the first arc-shaped portion to the second arc-shaped portion, wherein the second hook part includes: fourth and fifth arc-shaped portions respectively extending from the other side of the silicon body to symmetrical to each other, wherein each of the fourth and fifth arc-shaped portions extends in a tapered shape to allow the second hook part to be easily hooked on an area for surgery; and a first horizontal extending portion that horizontally extends from the fourth arc-shaped portion; a second horizontal extending portion that horizontally extends from the fifth arc-shaped portion to correspond to the first horizontal extending portion; and a connecting portion that connects the first horizontal extending portion to the second horizontal extending portion.

Also, the core encapsulated by the silicon body may further include a fourth bent portion that vertically connects the first bent portion to the third bent portion, and thus the core may have a ‘ [symbol] ’ shape.

Also, the silicon body may have a length of about 33 cm, and the first hook part may have a length of about 1.2 cm, and the second hook part may have a length of about 5.5 cm so that the area for surgery is easily hooked and pulled, and movement of the retractor is not interfered in a narrow space for surgery.

Also, the core may have at least one stepped portion on each of both sides thereof.

Also, the silicon body may further include a recessed portion having a diameter that is less than that thereof in one side thereof.

### BRIEF DESCRIPTION OF THE DRAWINGS

FIG. 1 is a perspective view of a retractor according to a related art.

FIG. 2 is a perspective of a core of a retractor for video-assisted thoracoscopic surgery according to the present invention.

FIG. 3 is a perspective view of the retractor for video-assisted thoracoscopic surgery according to an embodiment of the present invention.

FIG. 4 is a perspective view of a retractor for video-assisted thoracoscopic surgery according to another embodiment of the present invention.

FIG. 5 is a front view of the core of the retractor for video-assisted thoracoscopic surgery according to the present invention.

FIG. 6 is a front view of the retractor for video-assisted thoracoscopic surgery according to the present invention.

FIG. 7 is a cross-sectional view of the retractor for video-assisted thoracoscopic surgery according to the present invention.

FIG. 8 is a view of a first hook part of the retractor for video-assisted thoracoscopic surgery according to the present invention.

FIG. 9 is a view of a second hook part of the retractor for video-assisted thoracoscopic surgery according to the present invention.

FIG. 10 is a view of a retractor for video-assisted thoracoscopic surgery according to another embodiment of the present invention

FIG. 11 is a perspective view illustrating an example in which the retractor for video-assisted thoracoscopic surgery according to the present invention is freely bent.

FIG. 12 is a perspective view of a core of the retractor for video-assisted thoracoscopic surgery according to another embodiment of the present invention

FIG. 13 is a side view of the core of the retractor for video-assisted thoracoscopic surgery according to another embodiment of the present invention.

FIG. 14 is a perspective view of a retractor for video-assisted thoracoscopic surgery according to another embodiment of the present invention.

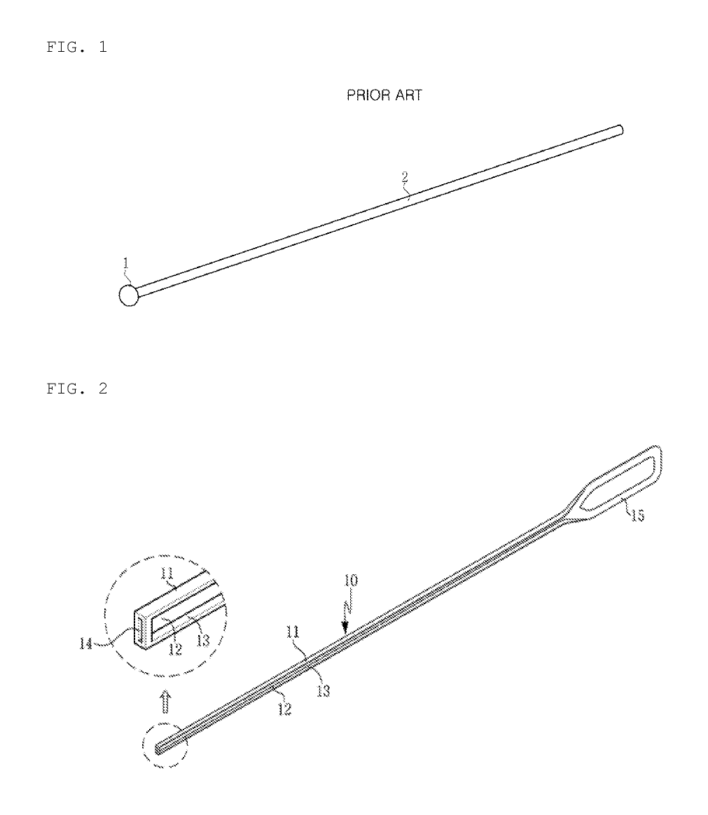

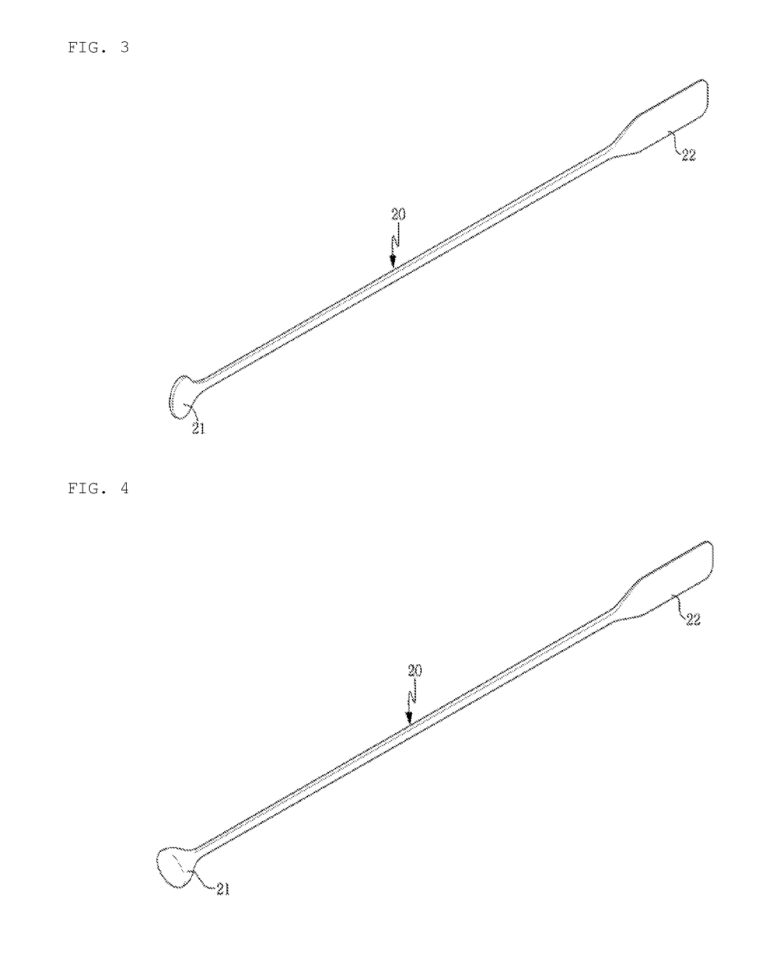

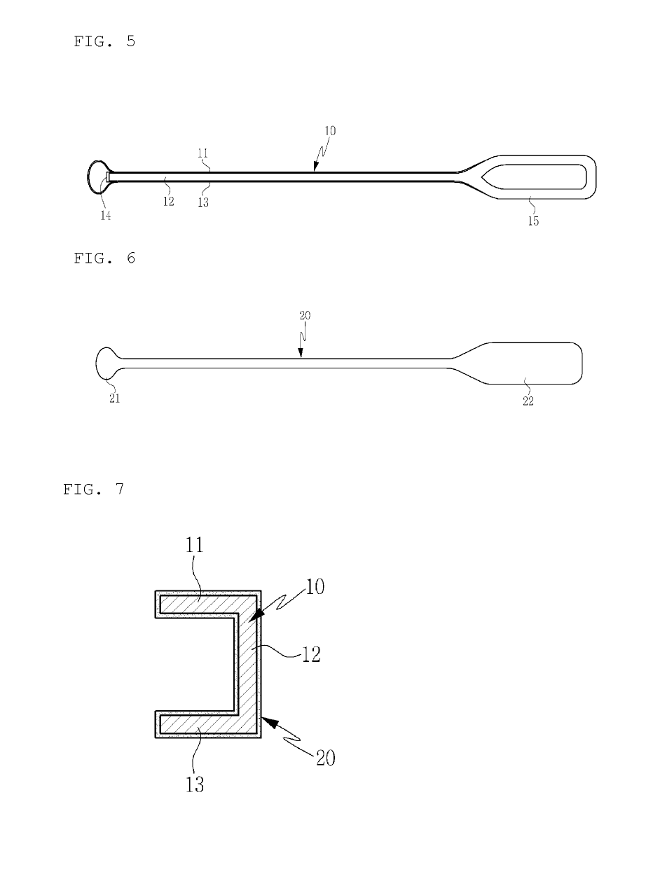

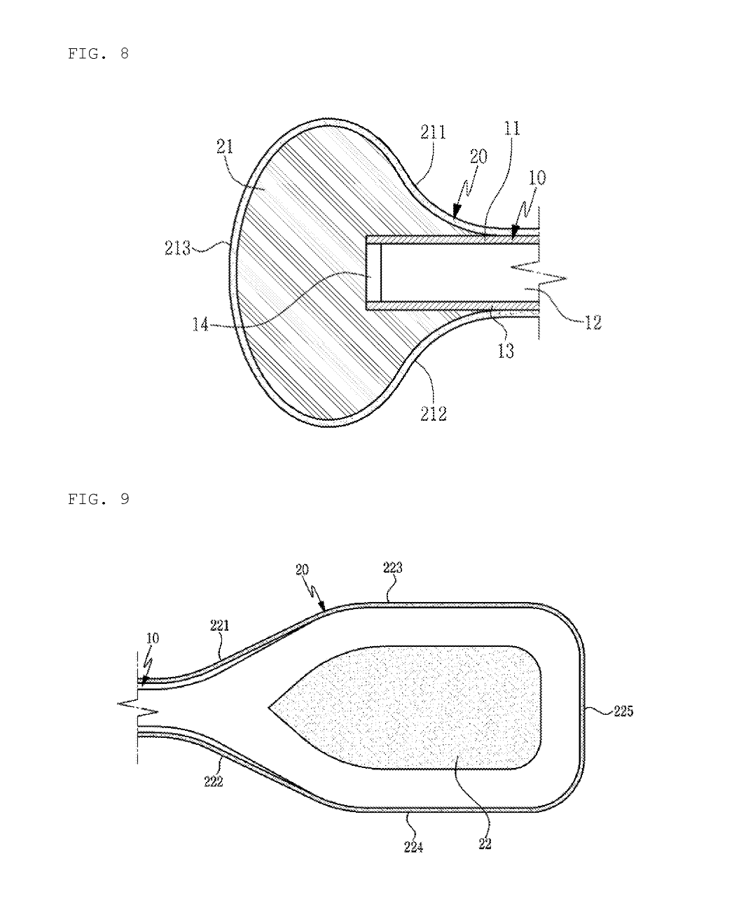

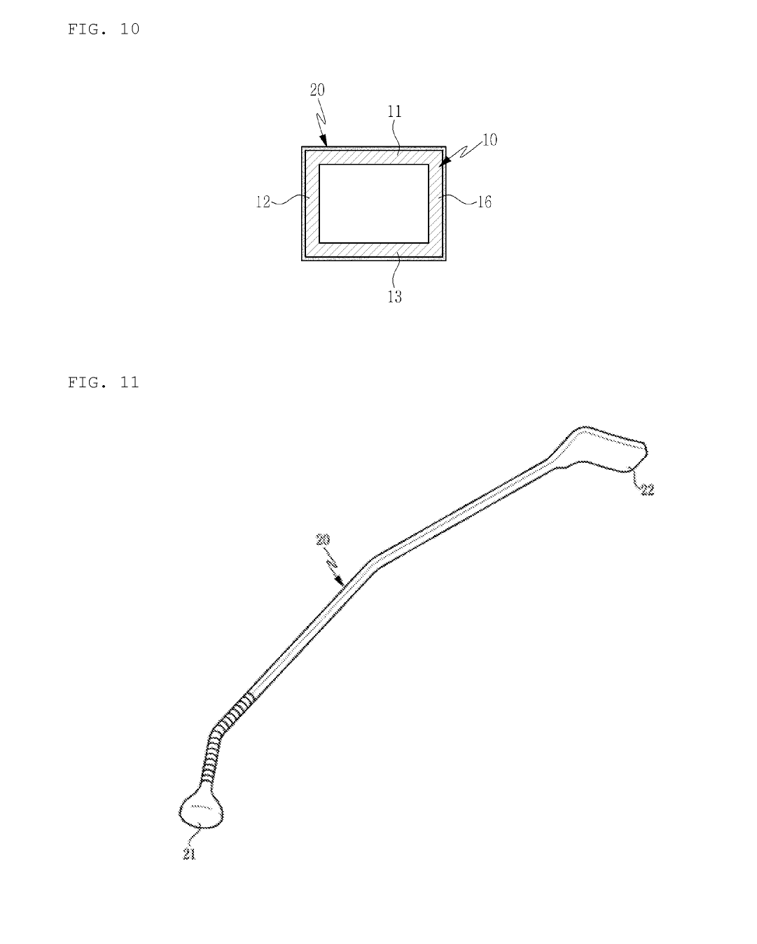

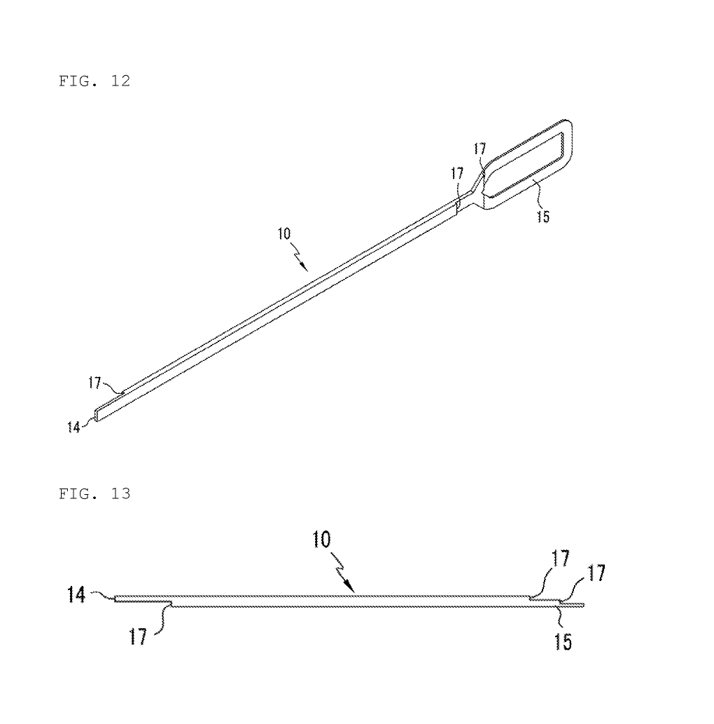

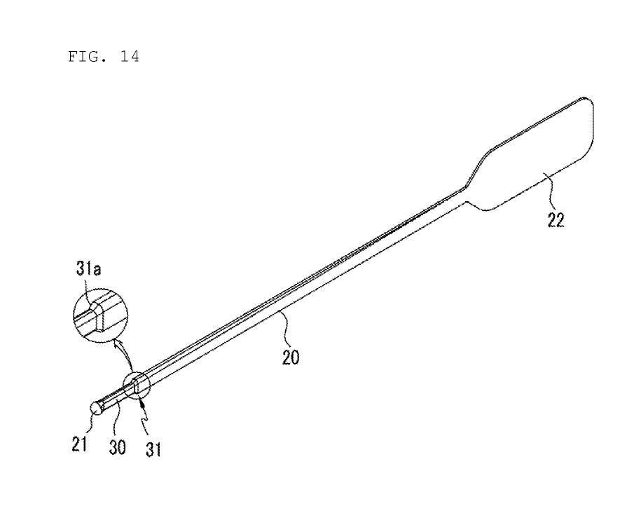

### DETAILED DESCRIPTION OF EXEMPLARY EMBODIMENTS

Reference will now be made in detail to the preferred embodiments of the present invention, examples of which are illustrated in the accompanying drawings.

Unless otherwise defined, all terms (including technical and scientific terms) used herein have the same meaning as generally understood by those skilled in the art. Terms as defined in a commonly used dictionary should be construed as having the same meaning as in an associated technical context, and unless defined apparently in the description, the terms are not ideally or excessively construed as having formal meaning.

Hereinafter, a retractor for video-assisted thoracoscopic surgery according to an embodiment of the present invention will be described in more detail with reference to the accompanying drawings.

As illustrated, a retractor for video-assisted thoracoscopic surgery according to the present invention includes a first bent portion 11, a second bent portion 12 vertically extending from the first bent portion 11, a third bent portion 13 horizontally extending from the second bent portion 12 to correspond to the first bent portion 11, a core 10 having an insert core part 14 on one side thereof and a hook core part 15 on the other side thereof, a silicon body 20 encapsulates a circumference of the core 10, a first hook part 21 extending from one side of the silicon body 20 to encapsulate the insert core part 14 of the core 10, and a second hook part 22 extending from the other side of the silicon body 20 to encapsulate the hook core part 15 of the core 10.

Since the core 10 is constituted by the first, second, and third bent portions 11, 12, and 13 extending to each other, the core 10 has a ‘ [symbol] ’ shape. Also, each of the first, second and third bent portions 11, 12, and 13 of the core 10 is formed of thin metal to be easily bent.

Also, since the core 10 is flexibly bent, and the silicon body 20 covers and encapsulates the circumference of the core 10, even though the core 10 is bent, a phenomenon in which an area for surgery tears or is damaged, or a glove for surgery tears due to the bent core 10 may be prevented.

Here, the core 10 may be formed of a stainless material and not be limited thereto. For example, the core 10 may be formed of various materials if the material is bendable.

As illustrated, the first and second hook parts 21 and respectively disposed on one and the other sides of the silicon body 20 according to the present invention may have structures and sizes different from each other so that one retractor is used when a view for surgery varying depending on the area for surgery is secured.

That is, according to the related art, since only one hook part is disposed on one side of the body, for example, when it is necessary to further secure a view for surgery during the surgery, another retractor has to be used, and as a result, various kinds of retractors respectively having various structures and sizes of hook parts have to be prepared. However, according to the present invention, since the first and second hook parts 21 and 22 having sizes and structures different from each other are respectively disposed on both sides of the silicon body 20 that covers and encapsulates an entire core 10, the first hook part 21 or the second hook part 22 may be selectively used as desired, and thus the reactor may be improved in usability.

Also, the core 10 may further include a fourth bent portion 16 that connects the first bent portion 11 to the third bent portion 13 so that the core 10 has a ‘ [symbol] ’ shape.

Here, the core 10 has a hollow inside. Like this, since the core 10 has a hollow inside, there is no inconvenience in bending the core 10 even though the core 10 has a ‘ [symbol] ’ shape.

Also, the core 10 according to another embodiment may further include at least one stepped portion 17 that is provided with a plate having a predetermined length on each of both sides thereof.

The stepped portion 17 is a constitution for easily bending the core 10. At least one stepped portion 17 is provided on the core 10. The stepped portion 17 may be formed by overlapping a plurality of thin plates in layers or by processing a thick plate into a stepped shape. When the stepped portion 17 is formed by overlapping the plurality of plates, a thickness of the core 10 may be accurately determined depending on required stiffness. When the thin plate is processed into a stepped shape, since it is unnecessary to perform welding and adhesion processes, the core 10 may be relatively easily manufactured and have relatively high stiffness because it is formed in a single unit.

Since the stepped portions 17 are provided on both sides of the core 10, the core 10 has both sides each of which has a thick thickness and a central portion having a predetermined thickness. Each of both sides of core 10 having a thin thickness due to the stepped portion 17 may be easily properly bent on the circumstance during the surgery. Since the central portion of the core 10 has a predetermined thickness, the central portion of the core 10 may be easily grasped.

Alternatively, two stepped portions 17 may be provided on the other side of the core 10 on which the hook core part 15 is disposed. Here, one of the two stepped portions 17 provided on the other side of the core 10 may be disposed on one side of the hook core part 15, and the other one may be disposed on a position at which the hook core part 15 contacts the first, second, and third bent portions 12, 13, and 14. Thus, since the hook core part 15 on which the two stepped portions 17 are disposed has a thick thickness, the hook core part 15 may be easily bent. Here, the core 10 may have a predetermined thickness from one side of the hook core part 15 to the portion at which the hook core part 15 contacts the first, second, and third bent portions 12, 13, and 14 to enable the core 10 to have stiffness enough to sustain the area for surgery during the surgery and a bendable thickness.

Instead of the stepped portions 17 on both sides of the core 10, an inclined part (not shown) that is inclined according to a difference of thickness of the core 10 is provided on the core 10 so that the core 10 has a consecutively connected shape. Since the core 10 has a consecutively connected shape by the inclined part, when the first or second hook parts 21 and 22 hooks or pulls the area for surgery, a phenomenon in which the area for surgery tears or is damaged by the stepped portion 17 may be prevented.

Alternatively, the stepped portion 17 may be provided on only one side of the core 10 but not be provided on both sides of the core 10.

The first hook part 21 according to the present invention has first and second arc-shaped portions 211 and 212 respectively symmetrically extending from the one side of the silicon body 20 that covers and encapsulates the entire core 10, which of each extends in a tapering shape so that each of the first and second arc-shaped portions 211 and 212 is easily hooked on the area for surgery and a third arc-shapes part 213 connecting the first arc-shaped portion 211 to the second arc-shaped portion 212.

Like this, since the first hook part 21 has the tapered first and second arc-shaped portions 211 and 212, the first hook part 21 may easily hook and pull the area for surgery by using one of the first and second arc-shaped portions 211 and 212.

This is done for preventing the circular retractor hook part according to the related art from sliding off the area for surgery. As illustrated, since each of the first and second arc-shaped portions 211 and 211 has a tapered arc shape protruding from the one side of the silicon body 20, a hooking operation on the area for surgery may be firmly performed, and as a result, even though the area for surgery is pulled, the retractor may not slide off the area for surgery.

Also, since each of the first and second arc-shaped portions 211 and 212 and the third arc-shaped portion 213 connecting the first arc-shaped portion 211 to the second arc-shaped portion 212 of the first hook part 21 has a curved arc shape as illustrated, when the area for surgery is pulled or hooked, a phenomenon in which the area for surgery tears or is damaged may be prevented.

The second hook part 22 according to the present invention has fourth and fifth arc-shaped portions 221 and 222 respectively symmetrically extending from the other side of the silicon body 20 that covers and encapsulates the entire core 10, which of each extends in a tapering shape so that each of the first and second arc-shaped portions 211 and 212 is easily hooked on the area for surgery and a first horizontal extending portion 223 horizontally extending from the fourth arc-shaped portion 221, a second horizontal extending portion 224 horizontally extending from the fifth arc-shaped portion 222 to correspond to the first horizontal extending portion 43, and a connecting portion 225 connecting the first horizontal extending portion 223 to the second horizontal extending portion 224.

Each of the fourth and fifth arc-shaped portions 221 and 222 may be hooked on the area for surgery to pull the area for surgery to secure a view of the area for surgery. Since the tapered fourth and fifth arc-shaped portions 221 and 222 are provided on the second hook part 22, the area for surgery may be easily hooked and pulled by using one of the fourth and fifth arc-shaped portions 221 and 222.

This is done for preventing the circular retractor hook part according to the related art from sliding off the area for surgery. As illustrated, since each of the fourth and fifth arc-shaped portions 221 and 222 has a tapered arc shape protruding from the other side of the silicon body 20, a hooking operation on the area for surgery may be firmly performed, and as a result, even though the area for surgery is pulled, the retractor may not slide off the area for surgery.

Also, since each of the fourth and fifth arc-shaped portions 221 and 222 and the connecting portion 225 of the second hook part 22 has a curved arc shape as illustrated, when the area for surgery is pulled or hooked, a phenomenon in which the area for surgery tears or is damaged may be prevented.

The silicon body 20 that covers and encapsulates the entire core 10 may have a length of about 33 cm, and the first hook part 21 may have a length of about 1.2 cm, and the second hook part 22 may have a length of about 5.5 cm so that the area for surgery is easily hooked and pulled, and movement of the retractor for video-assisted thoracoscopic surgery according to the present invention is not interfered in a narrow space for surgery.

Each of the silicon body 20, the first hook part 21, and the second hook part 22 is limited to the above-described length. Therefore, the retractor for video-assisted thoracoscopic surgery according to the present invention may freely move in a narrow space, i.e., a space for lung surgery and easily pull the area for surgery by using the first hook part 21 or the second hook part 22.

In a retractor for video-assisted thoracoscopic surgery according to another embodiment of the present invention, a recessed portion 30 having a diameter less than that of the silicon body 20 may be further defined in one side of the silicon body 20.

The recessed portion 30 is a constitution for allowing the retractor for video-assisted thoracoscopic surgery to be easily used in a narrower space. The recessed portion 30 has a diameter less than that of the silicon body 20 and is defined in one side of the silicon body 20. The recessed portion 30 may have a predetermined length defined in the silicon body 20 from the insert core part 14 disposed on the one end of the silicon body 20. Here, a protrusion portion 31 is formed on a boundary between the recessed portion 30 and the silicon body 20 by a diameter difference between the recessed portion 30 and the silicon body 20.

Since the recessed portion 30 has a diameter less than that of the silicon body 20, the recessed portion 30 may freely move in a narrow space for surgery to allow the retractor for video-assisted thoracoscopic surgery to be easily used in a narrow space for surgery.

The protrusion portion 31 is a constitution for allowing the retractor for video-assisted thoracoscopic surgery to be easily grasped. The protrusion portion 31 is formed on the boundary between the recessed portion 30 and the silicon body 20 by the diameter difference between the recessed portion 30 and the silicon body 20. When a user grasps the retractor for video-assisted thoracoscopic surgery, the protrusion portion 31 may prevent the user's hand from sliding due to a protruding shape thereof to allow the user to easily grasp the retractor for video-assisted thoracoscopic surgery during the surgery. Also, the protrusion portion 31 may have an inclined portion 31a that is inclined downward from the silicon body 20 to the recessed portion 30 to prevent the area for surgery from tearing or being damaged due to the protruding shape thereof when the area for surgery is pulled or hooked by the first hook part 21 or the second hook part 22.

The retractor for video-assisted thoracoscopic surgery not only be used in the video-assisted thoracoscopic surgery but also be used in a video-assisted laparoscopic surgery. Also, the retractor for video-assisted thoracoscopic surgery will not be limited to the above-described surgeries. For example, the retractor for video-assisted thoracoscopic surgery may be used other surgeries performed through a small incision.

The retractor according to the present invention may hook and pull the area for surgery such as a patient's lung, vessel, esophagus, and trachea/bronchus to secure a view of the area for surgery during the video-assisted thoracoscopic surgery and surround and protect tissues from galvanocautery occurring during the surgery. Also, the retractor may be bent during the video-assisted thoracoscopic surgery and thus easily hook and pull the area for surgery even in a narrow space and prevent an operator's view from being interfered therewith. Since the core is encapsulated by the silicon, when the area for surgery is pulled, the retractor may smoothly contact the area for surgery. Also, retracting parts having shapes different from each other may be disposed on both ends of the retractor to allow the retractor to be quickly reacted on the circumstances during the surgery.

The above detailed description exemplifies the present invention. Further, the above contents just illustrate and describe preferred embodiments of the present invention and the present invention can be used under various combinations, changes, and environments. That is, it will be appreciated by those skilled in the art that substitutions, modifications and changes may be made in these embodiments without departing from the principles and spirit of the general inventive concept, the scope of which is defined in the appended claims and their equivalents. The above-mentioned embodiments are used to describe a best mode in implementing the present invention. The present invention can be implemented in a mode other than a mode known to the art by using another invention and various modifications required a detailed application field and usage of the present invention can be made.

Therefore, the detailed description of the present invention does not intend to limit the present invention to the disclosed embodiments. Further, the appended claims should be appreciated as a step including even another embodiment.

What is claimed is:

1. A retractor for video-assisted thoracoscopic surgery, the retractor comprising:
a core provided with a first bent portion, a second bent portion vertically extending from the first bent portion, a third bent portion horizontally extending from the second bent portion to correspond to the first bent portion, the core having an insert core part on one side thereof and a hook core part on the other side;
a silicon body configured to encapsulate a circumference of the core;
a first hook part extending from one side of the silicon body to encapsulate the insert core part of the core; and
a second hook part extending from the other side of the silicon body to encapsulate the hook core part of the core,
wherein the first hook part comprises:
first and second arc-shaped portions respectively extending from the one side of the silicon body to symmetrical to each other, wherein each of the first and second arc-shaped portions extends in a tapered shape to allow the first hook part to be easily hooked on an area for surgery; and
a third arc-shaped portion that connects the first arc-shaped portion to the second arc-shaped portion,
wherein the second hook part comprises:
fourth and fifth arc-shaped portions respectively extending from the other side of the silicon body to symmetrical to each other, wherein each of the fourth and fifth arc-shaped portions extends in a tapered shape to allow the second hook part to be easily hooked on an area for surgery; and
a first horizontal extending portion that horizontally extends from the fourth arc-shaped portion;
a second horizontal extending portion that horizontally extends from the fifth arc-shaped portion to correspond to the first horizontal extending portion; and
a connecting portion that connects the first horizontal extending portion to the second horizontal extending portion.

2. The retractor of claim 1, wherein the core encapsulated by the silicon body further comprises a fourth bent portion that vertically connects the first bent portion to the third bent portion, and
the core has a ‘ [symbol] ’ shape.

3. The retractor of claim 1, wherein the silicon body has a length of about 33 cm, and the first hook part has a length of about 1.2 cm, and the second hook part has a length of about 5.5 cm so that the area for surgery is easily hooked and pulled, and movement of the retractor is not interfered in a narrow space for surgery.

4. The retractor of claim 1, wherein the core has at least one stepped portion on each of both sides thereof.

5. The retractor of claim 1, wherein the silicon body further comprises a recessed portion having a diameter that is less than that thereof in one side thereof.
